(19) United States
(12) Patent Application Publication
(10) Pub. No.: US 2012/0039983 A1
(43) Pub. Date: Feb. 16, 2012

(54) AMPHIPHILIC MACROMOLECULE-LIPID COMPLEXES

(76) Inventors: Kathryn E. Uhrich, New Brunswick, NJ (US); Alexander M. Harmon, New Brunswick, NJ (US)

(21) Appl. No.: 12/854,856

(22) Filed: Aug. 11, 2010

(51) Int. Cl. A61K 9/127; A61P 35/00; A61K 31/337

(52) U.S. Cl. 424/450; 514/449

(57) ABSTRACT

The invention provides amphiphilic macromolecule (AM)-lipid complexes, methods of making the complexes, and methods of using the complexes.

### BACKGROUND

Cationic lipids alone, or in combination with neutral lipids, have been used to form liposomes for use as delivery vehicles for therapeutic agents. While liposomes have been studied, issues including inherent cytotoxicity and instability under physiological conditions hinder their use as therapeutic delivery vehicles. Accordingly, improved therapeutic delivery vehicles are needed.

### SUMMARY OF CERTAIN EMBODIMENTS OF THE INVENTION

Certain embodiments of the present invention provide a complex that comprises an amphiphilic macromolecule (AM) and lipids.

In certain embodiments, the lipids comprise phospholipids.

In certain embodiments, the lipids comprise glycerophospholipids.

In certain embodiments, the lipids comprise cationic lipids, neutral lipids, or combinations thereof.

In certain embodiments, the lipids comprise C₁₆-C₂₀ lipids, e.g., C₁₈ lipids.

In certain embodiments, the complex comprises 1,2-diacyl-sn-glycero-3-phosphoethanolamine (DOPE), 1,2-diacyl-3-trimethylammonium-propane (DOTAP) 1,2-dioleoyl-sn-glycero-3-ethylphosphocholine (EPC) or 1,2-dioleoyl-sn-glycero-3-phosphocholine (DOPC), or a combination thereof.

In certain embodiments, the complex comprises the lipids 1,2-diacyl-sn-glycero-3-phosphoethanolamine (DOPE) and 1,2-diacyl-3-trimethylammonium-propane (DOTAP).

In certain embodiments, the complex is in the form of a micelle.

In certain embodiments, the complex is in the form of an AM-coated liposome.

In certain embodiments, the complex further comprises a therapeutic compound.

In certain embodiments, the therapeutic compound is a hydrophobic therapeutic compound.

In certain embodiments, the therapeutic compound is an anticancer drugs (e.g., doxorubicin, camptothecin), an immunosuppressive drug (e.g., cyclosporine), or an NSAID (e.g., indomethacin).

In certain embodiments, the therapeutic compound is paclitaxel.

In certain embodiments, the AM:lipid ratio is about 5:1.

In certain embodiments, the AM is a compound of formula (I):

A-X—Y—Z—R₁ (I)

wherein A is a carboxy group or is absent; X is a polyol, Y is —C(═O)—, —C(═S)—, or is absent; Z is O, S or NH; and R₁ is a polyether, wherein one or more hydroxy groups of the polyol are acylated with a fatty acid residue.

In certain embodiments, the polyol has from about 2 carbons to about 20 carbons.

In certain embodiments, the polyol is substituted with one or more carboxy groups.

In certain embodiments, the polyol is mucic acid, malic acid, citromalic acid, alkylmalic acid, hydroxy derivatives of glutaric acid, alkyl glutaric acids, tartaric acid, or citric acid.

In certain embodiments, the polyether is a poly(alkylene oxide) having between about 2 and about 150 repeating units.

In certain embodiments, the polyether is linked to the polyol through an ester, thioester, or amide linkage.

In certain embodiments, the polyether is a methoxy terminated poly(ethylene glycol).

In certain embodiments, the fatty acids comprise from about 2 to about 24 carbon atoms.

In certain embodiments, the AM is a compound of the following formula:

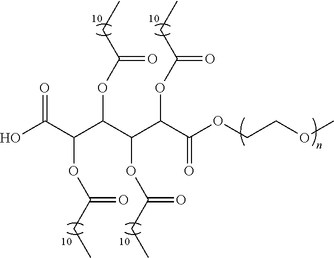

wherein n is an integer between 100 and 120.

Certain embodiments of the present invention provide a composition comprising a complex described herein.

Certain embodiments of the present invention provide a pharmaceutical composition comprising a complex described herein and a suitable carrier.

Certain embodiments of the present invention provide a method for delivering a therapeutic agent to an animal in need of treatment with the agent, comprising administering a complex described herein to the animal.

Certain embodiments of the present invention provide a method of making an amphiphilic macromolecule (AM)-lipid complex, comprising:

combining lipids and AMs in a solvent;

removing the solvent by evaporation to prepare a film;

drying the film; and

hydrated the film to produce a composition that comprises the complex.

In certain embodiments, the method further comprises filtering the composition to separate the complex.

Certain embodiments of the present invention provide a method of making an amphiphilic macromolecule (AM)-lipid complex, comprising:

adding the AM to a solution that comprises vesicles formed by the lipids so as form the AM-lipid complex.

Also provided are complexes prepared according to the methods described herein.

### BRIEF DESCRIPTION OF THE FIGURES

FIGS. 1A and 1B. AM-lipid complex in 10 mM HEPES buffer as assayed for size by DLS (1A) and turbidity by UV/Vis (1B). All complexes were assayed within 2-4 hours after production. Similar results were seen for complexes assayed up to seven days after production. Lipid content for all samples was fixed at 5 mg/ml. CE—co-evaporation, PA—post-addition.

FIG. 2. ITC of AM (6 mM=35.4 mg/ml) injected into 1 mM lipid (0.758 mg/ml). Small squares are integrated heat signals; larger squares indicate certain ratios of interest (AM/lipid=0.2, 0.5, 1, 5). The arrows denote two certain points of interest (AM/lipid=7 and 8).

FIG. 3 depicts a phase diagram.

FIG. 4 depicts the assembly of a PXT-loaded AM-lipid complex.

FIG. 5 depicts BT-20 cell viability after 72 hrs culture with PXT-loaded AM-lipid complexes. FIG. 5 illustrates that paclitaxel (PXT) loaded AM-lipid complexes, produced by CE, exhibit dose dependent cytotoxicity towards BT-20 intraductal carcinoma cell lines in vitro.

FIG. 6 depicts BT-20 cell viability after 72 hrs culture with PXT-loaded AM-lipid complexes FIG. 6 illustrates that paclitaxel (PXT) loaded AM-lipid complexes, produced by post addition (PA), exhibit dose dependent cytotoxicity towards BT-20 intraductal carcinoma cell lines in vitro.

### DETAILED DESCRIPTION

Amphiphilic co-polymers in combination with lipids were studied for drug delivery applications. Specifically, mucic acid-based amphiphilic macromolecules (AM) were complexed with, e.g., a 1:1 ratio of 1,2-dioleoyl-3-trimethylammonium-propane (DOTAP) and 1,2-dioleoyl-sn-glycero-3-phosphoethanolamine (DOPE) to form AM-lipid complexes. Altering the AM-lipid weight ratio and method of assembly allows the complexes to exist as either lamellar aggregates, micellar aggregates, or as AM-coated liposomes. The gradual increase in lipid ratios concurrently increased the zeta potential of the complexes, which directly correlates with increased cellular uptake of the complexes in vitro with increased uptake noted in BT-20 carcinoma cells verses normal fibroblasts. Increasing polymer content ratios increased complex steric stability in the presence of serum proteins and reduced the inherent cytotoxicity towards fibroblasts in vitro. AM-lipid complexes solublized paclitaxel and showed drug-mediated, dose-dependent cytotoxicity towards target BT-20 cells in vitro. AM-lipid complexes are good tools as drug delivery systems due to their tunable zeta potential and cellular uptake, steric stability, inherently low cytotoxicity, and ability to load and deliver insoluble chemotherapeutic agents.

AMs combined with DOPE:DOTAP by co-evaporation methods produce AM-lipid complexes existing as lamellar aggregates at low AM concentration or micellar aggregates at high AM concentration. AMs combined with DOPE:DOTAP by post-addition methods produce AM-lipid complexes existing as AM-coated liposomes. AM-lipid complexes prepared by both methods exhibit optimal drug delivery system attributes, including a tunable zeta potential which correlates to a tunable cellular uptake, increased uptake in carcinoma cell lines verses normal cell lines, steric stability, inherently low cytotoxicity, and the ability to load and deliver insoluble chemotherapeutic agents. Paclitaxel-loaded AM-lipid complexes exhibit cytotoxicity in vitro towards target carcinoma cell lines in a dose-dependent manner

AM-lipid complexes can be used as carriers for therapeutic agents such as, but not limited to, hydrophobic anti-cancer drugs. An example of a hydrophobic anticancer drug is paclitaxel.

AM-lipid complexes include cationic liposomes coated with amphiphilic macromolecules. These complexes improve inherent drug delivery deficiencies of liposomes, such as reducing their inherent non-specific cytotoxicity and poor steric stability while maintaining their high drug loading capacity and cellular uptake. AM-lipid complexes may show a preferential uptake in carcinoma cell lines verses normal cell lines, which may represent a passive targeting system. Paclitaxel-loaded AM-lipid complexes showed increased tolerability in mice when compared to the current commercial paclitaxel formulation (TAXOL®). This difference in tolerability should allow for increased clinical efficacy of paclitaxel-based chemotherapies.

An example of an AM is an AM of the following formula:

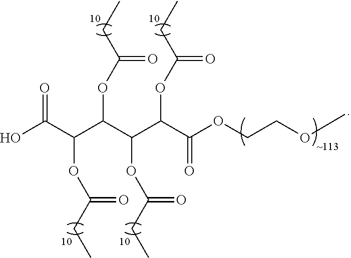

Other examples of AMs are found in International Publication Number WO 03/103594; U.S. Pat. No. 7,470,802, e.g., as compounds of formula I. AMs can be about 10-30 nm in diameter.

Certain AMs are described by compounds of formula (I):

A-X—Y—Z—R₁ (I)

wherein A is a carboxy group or is absent; X is a polyol, Y is —C(═O)—, —C(═S)—, or is absent; Z is O, S or NH; and R₁ is a polyether, wherein one or more hydroxy groups of the polyol are acylated with a fatty acid residue, will aggregate in a solvent to form micellar structures.

Additionally, compounds of formula (I) having unsaturated bonds (e.g., in the fatty acid or polyether groups), can be cross-linked after aggregate formation to form covalently bound structures (i.e., cross-linked micelles). These aggregates, both cross-linked and uncross-linked, are useful in drug delivery applications, as well as in many other applications where traditional micelles and AMs can be applied. The aggregates formed from compounds of formula (I), both cross-linked and uncross-linked, can be prepared.

A is a carboxy group or is absent. When present, A may optionally be substituted with or attached to a bioactive or therapeutically active molecule. In one embodiment, the bioactive or therapeutically active molecule is vitamin E, sulfonic acids, sulfonates, or salicylic acid.

As used herein the term “polyol” includes straight chain and branched chain aliphatic groups, as well as mono-cyclic and poly-cyclic aliphatics, which are substituted with two or more hydroxy groups. A polyol typically has from about 2 carbons to about 20 carbons; preferably, from about 3 carbons to about 12 carbons; and more preferably from about 4 carbons to about 10 carbons. A polyol also typically comprises from about 2 to about 20 hydroxy groups; e.g., from about 2 to about 12 hydroxy groups; and e.g., from about 2 to about 10 hydroxy groups. A polyol can also optionally be substituted on a carbon atom with one or more (e.g., 1, 2, or 3) carboxy groups (COOH). These carboxy groups can conveniently be used to link the polyol to the polyether in a compound of formula (I).

One specific polyol is a mono- or di-carboxyllic acid containing from 1 to about 10 carbon atoms and substituted with from 1 to about 10 hydroxyl groups. The mono- or di-carboxylic acid may be a straight chained or branched chained aliphatic, or a mono-cyclic or poly-cyclic aliphatic compound. Suitable dicarboxylic acids include mucic acid, malic acid, citromalic acid, alkylmalic acid, hydroxy derivatives of glutaric acid, and alkyl glutaric acids, tartaric acid, citric acid, hydroxy derivatives of rumadic acid, and the like. Suitable monocarboxylic acids include 2,2-(bis(hydroxymethyl)propionic acid, and N-[tris(hydroxymethyl)methyl]glycine (tricine).

Another specific polyol is a “saccharide,” which includes monosaccharides, disaccharides, trisaccharides, polysaccharides and sugar alcohols. The term includes glucose, sucrose fructose and ribose, as well as deoxy sugars such as deoxyribose and the like. Saccharide derivatives can conveniently be prepared by methods known to the art. Examples of suitable mono-saccharides are xylose, arabinose, and ribose. Examples of di-saccharides are maltose, lactose, and sucrose. Examples of suitable sugar-alcohols are erythritol and sorbitol.

As used herein, the term polyether includes poly(alkylene oxides) having between about 2 and about 150 repeating units. Typically, the poly(alkylene oxides) have between about 50 and about 110 repeating units. The alkylene oxide units contain from 2 to 10 carbon atoms and may be straight chained or branched. The alkylene oxide units may contain from 2 to 10 carbon atoms. Poly(ethylene glycol) (PEG) is one embodiment. Alkoxy-, amino-, carboxy-, and sulfo-terminated poly(alkylene oxides) are preferred, with methoxy-terminated poly(alkylene oxides) are also embodiments.

A certain polyether has the following structure:

R₅—(R₆—O—)ₐ—R₆-Q—

wherein R₅ is a 1 to 20 carbon straight-chain or branched alkyl group, —OH, —OR₇, —NH₂, —NHR₇, —NHR₇R₈, —CO₂H, —SO₃H (sulfo), —CH₂—OH, —CH₂—OR₇, —CH₂—O—CH₂—R₇, —CH₂—NH₂, —CH₂—NHR₇, —CH₂—NR₇R₈, —CH₂CO₂H, —CH₂SO₃H, or —O—C(═O)—CH₂—CH₂—C(═O)—O—;

R₆ is a 1 to 10 carbon straight-chain or branched divalent alkylene group;

each R₇ and R₈ is independently a 1 to 6 carbon straight-chain or branched alkylene group;

Q is —O—, —S—, or —NR₇; and

a is an integer from 2 to 150, inclusive.

Another polyether is methoxy terminated polyethylene glycol.

In a compound of formula (I), a poly(alkylene oxide) can be linked to a polyol, for example, through an ether, thioether, amine, ester, thioester, thioamide, or amide linkage. A poly(alkylene oxide) may be linked to a polyol by an ester or amide linkage in a compound of formula (I).

As used herein, the term fatty acid includes fatty acids and fatty oils as conventionally defined, for example, long-chain aliphatic acids that are found in natural fats and oils. Fatty acids typically comprise from about 2 to about 24 carbon atoms. Preferably, fatty acids comprise from about 6 to about 18 carbon atoms. The term “fatty acid” encompasses compounds possessing a straight or branched aliphatic chain and an acid group, such as a carboxylate, sulfonate, phosphate, phosphonate, and the like. The “fatty acid” compounds are capable of “esterifying” or forming a similar chemical linkage with hydroxy groups on the polyol. Examples of suitable fatty acids include caprylic, capric, lauric, myristic, myristoleic, palmitic, palmitoleic, stearic, oleic, linoleic, eleostearic, arachidic, behenic, erucic, and like acids. Fatty acids can be derived from suitable naturally occurring or synthetic fatty acids or oils, can be saturated or unsaturated, and can optionally include positional or geometric isomers. Many fatty acids or oils are commercially available or can be readily prepared or isolated using procedures known to those skilled in the art.

Additional amphiphilic macromolecules that can be used use for the creation of AM-lipid complexes include, but are not limited to, tartaric acid-based AMs.

Additional lipids can be used for the creation of AM-lipid complexes include, but are not limited to, 1,2-dioleoyl-sn-glycero-3-ethylphosphocholine (EPC) and 1,2-dioleoyl-sn-glycero-3-phosphocholine (DOPC).

The complexes are useful for solubilizing hydrophobic molecules, particularly therapeutic agents that are hydrophobic in nature.

The complexes of the invention that comprise a therapeutic agent can be formulated as pharmaceutical compositions and administered to a mammalian host, such as a human male or female patient in a variety of forms adapted to the chosen route of administration, e.g., orally or parenterally, by intravenous, intramuscular, topical or subcutaneous routes. In certain embodiments, the complexes will be administered via solution, e.g., nasally, in eye drops, or via injection, and not orally.

The complexes of the invention may be administered intravenously or intraperitoneally by infusion or injection. Solutions of the complexes can be prepared, for example, in water. Under ordinary conditions of storage and use, these preparations may contain a preservative to prevent the growth of microorganisms.

The pharmaceutical dosage forms suitable for injection or infusion should be sterile, fluid and stable under the conditions of manufacture and storage. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride.

Sterile injectable solutions are prepared by incorporating the complexes of the invention in the required amount in the appropriate solvent with various of the other ingredients enumerated above, as required, followed by sterilization.

The dose and method of administration will vary from animal to animal and be dependent upon such factors as the type of animal being treated, its sex, weight, diet, concurrent medication, overall clinical condition, the particular therapeutic agent employed, the specific use for which the agent is employed, and other factors which those skilled in the relevant field will recognize.

Therapeutically effective dosages may be determined by either in vitro or in vivo methods. For each particular dosage form of the present invention, individual determinations may be made to determine the optimal dosage required. The range of therapeutically effective dosages will naturally be influenced by the route of administration, the therapeutic objectives, and the condition of the patient. The determination of effective dosage levels, that is, the dosage levels necessary to achieve the desired result, will be within the ambit of one skilled in the art. Typically, applications of agent are commenced at lower dosage levels, with dosage levels being increased until the desired effect is achieved.

A typical dosage might range from about 0.001 mg to about 1,000 mg of therapeutic agent, per kg of animal weight. Preferred dosages range from about 0.01 mg/kg to about 100 mg/kg, and more preferably from about 0.10 mg/kg to about 20 mg/kg. Advantageously, the dosage forms of this invention may administered several times daily, and other dosage regimens may also be useful.

Additional drugs for use with AM-lipid complexes include but are not limited to anticancer drugs (doxorubicin, camptothecin), immunosuppressive drugs (cyclosporine), and NSAIDs (indomethacin). Additional therapeutics for use with AM-lipid complexes include but are not limited to nucleic acids, proteins, macromolecules, and anti-bodies.

Certain embodiments of the invention will now be illustrated by the following non-limiting Examples. Certain embodiments are also described in the following: Lash et al., “Amphiphilic Macromolecule-Lipid Complexes for Controlled Delivery of Chemotherapeutic Drugs”, NJACS Polymer Topical Group and Controlled Release Society: Polymers in Drug Delivery Symposium, Rutgers University, Piscataway, N.J., (2009); Harmon et al., “Amphiphilic Macromolecule-Lipid Complexes For Use In Drug Delivery”, Baekeland Symposium, Rutgers University, Piscataway, N.J.; (2009); and Harmon et al., “Complexation of Amphiphilic Macromolecule (AM) with Cationic and Neutral Lipids for Improved Cellular Interactions and Therapeutic Delivery”, ACS national meeting, Washington, D.C.; (2009).

### Example 1

### Interaction of Amphiphilic Macromolecules with Cationic Liposomes

Surfactants are soluble amphiphilies that commonly form micelles in aqueous solution. When mixed with lipid bi-layer structures, such as small unilamellar vesicles (SUV), the surfactant solublizes the vesicle from a lamellar structure to an intermediate structure, and finally to mixed micelle structure. The structure formed is mediated by the ratio of the surfactant to lipid, with increasing surfactant leading to increased solublization. Solublization is achieved by surfactant molecules partitioning the lipid bi-layer structure. The onset of solublization, R^{sat}, occurs with a saturation of the lipid bi-layer structure and correlates with a morphological change from a lamellar structure to an intermediate structure. With increasing surfactant concentration in the lipid bi-layer, complete solublization, R^{sol}, is achieved. Complete solublization correlates with a morphological change from an intermediate structure to a mixed micelle. This solublization phenomenon occurs both when a surfactant solution is added to a vesicle solution and when the two components are combined in a common organic solvent, the solvent removed by evaporation, and the heterogeneous film hydrated in aqueous solution. Physio-chemical characteristics such as size and turbidity of lamellar, intermediate, and micellar aggregates are distinct with micellar aggregates exhibiting a distinct decrease in size and turbidity as compared to lamellar aggregates.

### AM-Lipid Complexes

Amphiphilic macromolecules (AMs) are non-cytotoxic, non-immunogenic, biodegradable materials that self-assemble at low critical micelle concentrations to form micelles (Harmon et al., Journal of Bioactive and Compatible Polymers, 24 185-197 (2009); Djordjevic et al., Pharm Res, 22(1), 24-32 (2005); Wang et al., Journal of Bioactive and Compatible Polymers, 21, 297-313 (2006); Liu et al., J Polymer Sci: Part A: Polym Chem, 37(6), 703-712 (1999); Guo et al., Implants and Tissue Engineering, 550, Materials Research Society Symposium Proceedings, Pittsburgh, 89-94 (1999); Liu et al., Journal of Controlled Release, 68, 167-174 (2000); Tao et al., J Colloid Interf Sci, 298, 102-110 (2006); Tian et al., Macromolecules, 37(2), 538-543 (2004)). The AMs are similar to conventional polymeric micelles, existing as thermodynamic aggregates with inherent thermodynamic instability (Djordjevic et al., Pharm Sci, 5(4), 256-267 (2003)). However, unlike conventional polymeric micelles, they have high solution stability and form significantly smaller micelles.

AMs were electrostatically complexed with a 1:1 ratio of 1,2-dioleoyl-3-trimethylammonium-propane (DOTAP) and 1,2-dioleoyl-sn-glycero-3-phosphoethanolamine (DOPE) to form AM-lipid complexes. These complexes are useful for delivering drugs, such as chemotherapeutic drugs. The complexes were produced by the addition of formed AM micelles in aqueous solution to formed small unilamellar vesicles in solution. Using this post-addition method, the size of the complex remains relatively consistent with the liposome alone. Therefore, the complex is considered to exist as a AM-coated liposome. The properties of the complexes can be optimized for drug delivery applications by altering the AM-lipid weight ratio. AM-lipid exhibit a tunable zeta potential, steric stability, inherently low cytotoxicity, and ability to load and deliver insoluble chemotherapeutic agents. In vitro, AM-lipids exhibited a preferential uptake in carcinoma cells over normal cells demonstrates a unique, passive targeting approach to delivery anti-cancer therapeutics.

In addition to the post addition method of production, a co-evaporation method was utilized to produce AM-lipid complexes. This method allows the hydrophobic domain of AMs to localize and embed into the lipid hydrocarbon chain bilayer during vesicle formation upon hydration of dried AM-lipid films. The AM's hydrophilic PEG would then be tethered to the bilayer and impart steric stability to the lamellar aggregate. AM-lipid complexes formed by this method have been found to be closer to equilibrium than other methods of assembly.

### Material and Methods

### Materials

1,2-Dioleoyl-3-trimethylammonium-propane (DOTAP) and 1,2-dioleoyl-sn-glycero-3-phosphoethanolamine (DOPE) were obtained from Avanti Polar Lipid (Alabaster, Ala.). Non-functionalized AMs were prepared as previously described (Tian et al., Macromolecules, 37(2), 538-543 (2004)). All other solvents and reagents, unless otherwise stated, were purchased from Fisher Scientific (Pittsburgh, Pa.) and Sigma-Aldrich (St. Louis, Mo.) and used as received.

### Preparation of AM-Lipid Complexes

AM-lipid complexes were prepared as weight-to-weight mixtures of AM and total weight of DOPE:DOTAP (1:1) lipids. The ratio of the lipids may be varied from the 1:1 ratio. The AM: lipid weight ratios assayed were 0:1, 1:10, 1:5, 1:2, 1:1, 2:1, 5:1, 10:1 and 1:0. AM-lipid complexes were created by either a co-evaporation (CE) (Clani et al., Biochim Biophys Acta, 1664(1), 70-79 (2004)) or a post-addition (PA) method (see below).

In the CE method, separate stock solutions of lipids in chloroform and AM in chloroform were used. Aliquots were co-dispensed into glass vials and solvent removed by rotary evaporation. The remaining film was dried overnight under vacuum at room temperature. The films were hydrated with 10 mM HEPES buffer and incubated on a rotary shaker overnight at room temperature. Materials were then processed through eight freeze-thaw cycles by immersion in a dry ice-methanol bath followed by immersion in a water bath at 60° C. Materials were then passed 27 times through a 100 nm filter using a mini-extruder (Avanti Polar Lipids). Processed materials were stored at 4° C. until use. All samples were used within four to seven days of production.

In the PA method, DOPE:DOTAP (1:1) small unilamellar vesicles (SUV) were first formed using the co-evaporation methods described above but without AMs present. Dried AM powder stock was hydrated in 10 mM HEPES. Concentrated aliquots of pre-formed SUV were added to concentrated aliquots of AM in 10 mM HEPES and brought to desired volume. Samples were statically incubated for at least one-hour at 25° C. Processed materials were stored at 4° C. until use. All samples used within four to seven days of production.

### Complex Size by Dynamic Light Scattering

AM-lipid complexes prepared from both methods were assayed for size by dynamic light scattering (DLS) using a NanoZS90 Instrument (Malvern Instruments, Malvern, UK). One milliliter samples were evaluated at least 1 mg/ml total AM-lipid complex in 10 mM HEPES at room temperature. Values reported represent the average of three runs with ten measurements per run for each sample.

### Turbidity

AM-lipid complexes prepared from both methods were assayed for turbidity by measuring percent transmission using an UV/Vis spectrophotometer. (Perkin Elmer Lambda XLS, Beaconsfield, UK). 400-600 nm wavelength scans were performed with percent transmission at 500 nm (Goni et al., Biochim Biophys Acta, 1508, 51-68 (2000), Karlovska et al., Gen Physiol Biophys, 26(4), 290-297 (2007)). One milliliter samples were evaluated at least 1 mg/ml total AM-lipid complex in 10 mM HEPES at room temperature. 10 mM HEPES was used as a blank prior to sample analysis. Values reported represent the average of three measurements per run for each sample.

### Results and Discussion

### AM-Lipid Size and Turbidity

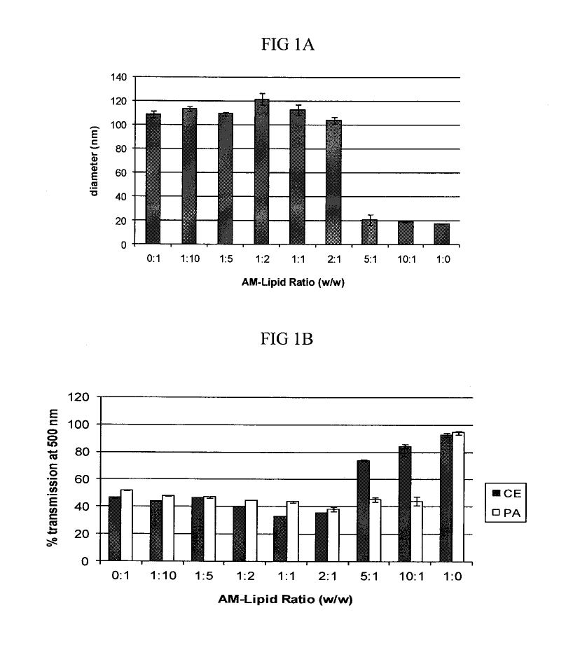

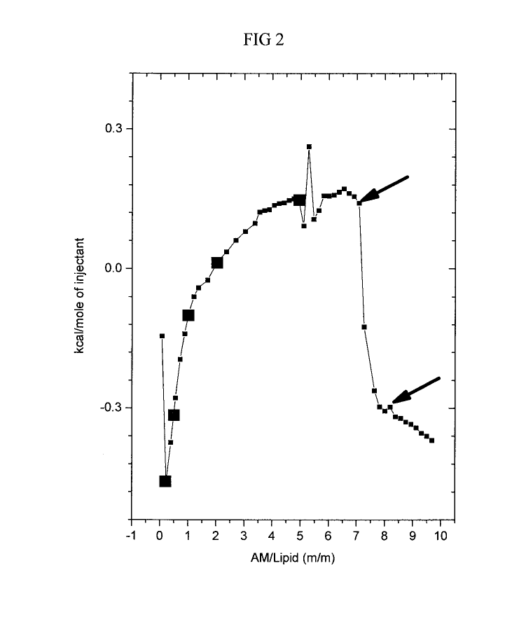

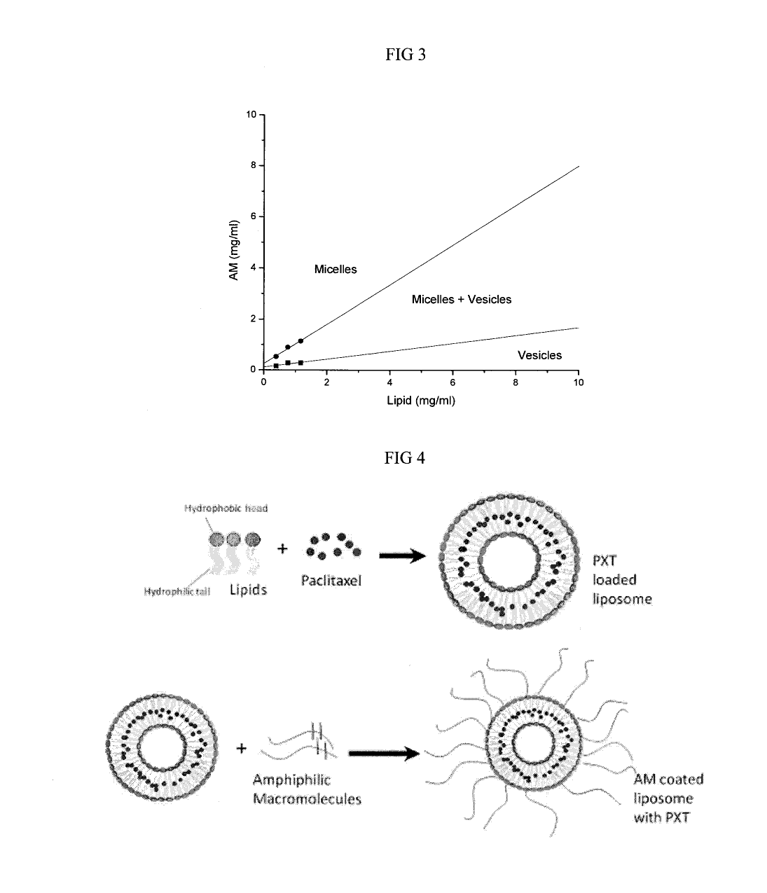

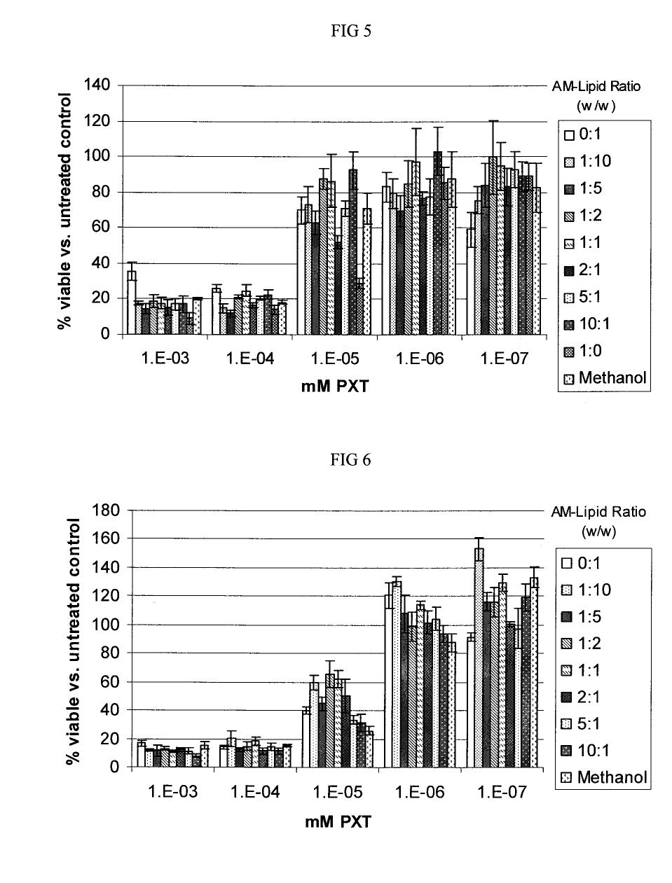

FIG. 1 shows the size of the AM-lipid complexes as assayed by DLS. The AM alone values (˜20 nm) were consistent with previous data and the liposome alone values (˜100 nm) were consistent with the 100 nm filter used in the extrusion process. DLS histograms for all samples produced by both co-evaporation and post addition showed a single peak curve with low standard deviation, indicating the AM and lipid combined to form a distinct, single structure. Complexes produced by the co-evaporation method maintained a size consistent with an extruded liposome at low AM concentrations. However, as AM concentration was increased to an AM-lipid ratio of 5:1, the size of the complexes sharply decreased to a size consistent with the AM micelle alone.

The turbidity of AM-lipid complexes was evaluated to determine if a given AM-lipid weight ratio existed as a lamellar or micellar structure. FIG. 1B shows the turbidity of the AM-lipid complexes as expressed by the percent transmission at 500 nm as measured by UV/vis spectroscopy. The turbidity values of the lipid alone (˜45%) and AM alone (˜95%) are consistent with the turbidity characteristics of liposomes and micelles respectively. Similar to the DLS data, AM-lipid complexes produced by post-addition were consistent with the lipid alone sample. Complexes produced by the co-evaporation method maintain a turbidity that is consistent with that of a lipid alone at low AM concentrations with a slight increase in turbidity noted with increased AM concentration. This data corresponds with the known concept that surfactants induce and increase liposome turbidity prior to their complete solubilization to mixed micelles. As AM concentrations increases to an AM-lipid ratio of 5:1, complex size sharply decreased to a turbidity consistent with the AM micelle alone.

### Example 2

### Micelle-Lipid Complexes Used to Improve Drug Delivery

Micelle-forming amphiphilic scorpion-like macromolecules (AScMs) can be used for medicinal applications, such as for the systemic delivery of hydrophobic anti-cancer drugs. While a drug-loaded micelle may increase the drug's solubility and stability in vivo, as described herein, a combined micelle-lipid complex will improve its delivery applications. Specifically, tailoring the ratios of DOPE (1,2-diacyl-sn-glycero-3-phosphoethanolamine) a neutral lipid, and DOTAP (1,2-diacyl-3-trimethylammonium-propane) a cationic lipid, can increase the drug's efficacy.

Micelle-lipid complexes can exist as mixed micelles or as polymer-coated liposomes, depending on the polymer lipid weight ratio and method of assembly. By gradually adding lipids to AScMs, the zeta potential of the complex is increased in a controlled fashion. This complex allows for tunable zeta potentials that can be used to optimize cellular uptake. A design of experiments used to evaluate optimal polymer-lipid and material-drug ratios for drug loading and in vitro cytotoxicity showed that a high lipid content had the greatest effects on drug loading efficiency and high material-drug ratios had the greatest effects on in vitro cytotoxicity. This work indicates that micelle-lipid complexes can be designed to optimize the efficacy of drugs, such as chemotherapeutic agents.

An AScM was used to aid in the delivery of drugs, such as Paclitaxel (PXT), to human breast cancer cells. The AM was composed of mucic acid with 12-carbon acyl chains and poly(ethelyene glycol) (PEG) of 5000 molecular weight. Since the mucic acid component of was highly hydrophobic and the PEG is highly hydrophilic, this AScM formed micelles.

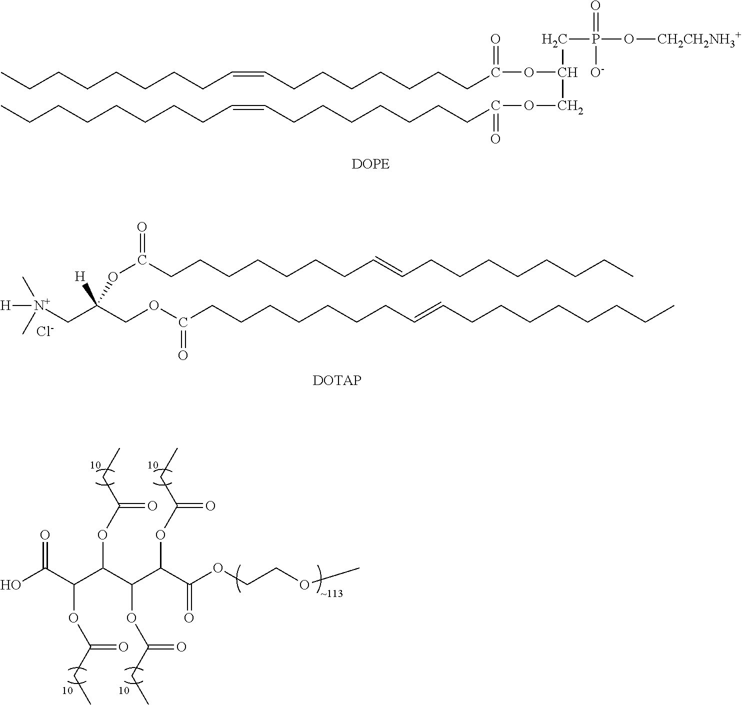

The micelles alone have high stability and biocompatibility, but poor drug loading efficiency. Comparatively, liposomes alone have high loading efficiencies but poor stabilization. Polymer-lipid complexes were investigated to determine if the high stability properties could be combined with the high loading efficiency of liposomes.

Also, increased nanocarrier zeta potentials can correspond with increased cellular uptake and therefore better delivery of drug-loaded nanocarriers. (Wang et al., Journal of Medicinal Chemistry, 41, 2207-2215 (1998)). The effects of altering the ratio of DOPE to DOTAP, and ultimately the ratio of polymer to lipid, were investigated to tune the zeta potential of the polymer-lipid complex.

### Materials and Methods

The AM was synthesized using previously described methods. DOPE and DOTAP were purchased from Avanti Polar Lipids.

Polymer and lipid complexation involved evaporating the polymer and lipid solvent and dry film rehydration followed by processing through 8 freeze-thaw cycles and filtration to 100 nm using an Avanti Polar Lipids mini extruder.

Drug loading involved the addition of PXT in methanol to the polymer-lipid complex during solvent evaporation. The product was processed as described above. Optimal drug loading and cytoxicity were evaluated through an extreme mixture design of experiments (DOE). In vitro cytotoxicity was determined by treating in vitro BT-20 intraductal carcinoma cells with drug loaded materials for 72 hours. Testing for size and zeta potential was performed using Dynamic Light Scattering (DLS), drug loading was evaluated by UV/vis spectrophotometry, and cell viability was evaluated by MTS assay.

### Results

Regression analysis of DOE showed that lipids, followed by PXT, followed by polymer had the most significant effect on the complex (R-sq=98.13%). High (30:1) material-drug ratios had increased loading efficiency vs. low (10:1) ratios.

Untreated materials remained stable at 37° C. It was determined that increased polymer content correlates with a decrease in size and increased solution translucence inferring a solublization of the liposome to a mixed-micelle. It was determined that increasing the amount of polymer increases stability in the presence of fetal bovine serum. It was determined that the polymer reduces liposome flocculation, inferring increase steric stability of the liposome.

Regression analysis of DOE showed that high drug loading ratios had the most significant impact on drug cytotoxicity, while the effect of polymer and lipid concentrations was equal (R-sq=79.87%). A 30:1 material drug ratio showed increased cytotoxicity verses 10:1 ratios at equal drug concentrations.

The zeta potential increased with the addition of lipid to polymer. The following ratios were evaluated: 0:1, 1:10, 1:5, 1:2, 1:1, 2:1, 5:1, 10:1 and 1:0.

Thus, gradual addition of lipid to polymer increases zeta potential in a controlled fashion. High material-drug ratios corresponded to increased drug loading efficiency and increased cytoxicity at equal drug concentrations than low material-drug ratios. Polymers increase the stability of polymer-lipid complexes in presence of serum protein. Accordingly, AScM-DOPE:DOTAP complexes, such as complexes, can be used to increase drug delivery and efficacy verses AScM or DOPE:DOTAP alone.

All publications cited herein are incorporated herein by reference. While in this application certain embodiments of invention have been described, and many details have been set forth for purposes of illustration, it will be apparent to those skilled in the art that certain of the details described herein may be varied without departing from the basic principles of the invention.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not pose a limitation on the scope of the invention unless otherwise specifically recited in the claims. No language in the specification should be construed as indicating that any non-claimed element as essential to the practice of the invention.

What is claimed is:

1. A complex that comprises an amphiphilic macromolecule (AM) and lipids.

2. The complex of claim 1, wherein the lipids comprise phospholipids.

3. The complex of claim 1, wherein the lipids comprise glycerophospholipids.

4. The complex of claim 1, wherein the lipids comprise cationic lipids, neutral lipids, or combinations thereof.

5. The complex of claim 1, wherein the lipids comprise C₁₆-C₂₀ lipids.

6. The complex of claim 1, wherein the complex comprises 1,2-diacyl-sn-glycero-3-phosphoethanolamine (DOPE), 1,2-diacyl-3-trimethylammonium-propane (DOTAP) 1,2-dioleoyl-sn-glycero-3-ethylphosphocholine (EPC) or 1,2-dioleoyl-sn-glycero-3-phosphocholine (DOPC), or a combination thereof.

7. The complex of claim 5, wherein the complex comprises the lipids 1,2-diacyl-sn-glycero-3-phosphoethanolamine (DOPE) and 1,2-diacyl-3-trimethylammonium-propane (DOTAP).

8. The complex of claim 1, wherein the complex is in the form of a micelle.

9. The complex of claim 1, wherein the complex is in the form of an AM-coated liposome.

10. The complex of claim 1, wherein the complex further comprises a therapeutic compound.

11. The complex of claim 10, wherein the therapeutic compound is a hydrophobic therapeutic compound.

12. The complex of claim 10, wherein the therapeutic compound is an anticancer drug, an immunosuppressive drug, or an NSAID.

13. The complex of claim 10, wherein the therapeutic compound is paclitaxel.

14. The complex of claim 1, wherein the AM:lipid ratio is about 5:1.

15. The complex of claim 1, wherein the AM is a compound of formula (I):
A-X—Y—Z—R₁ (I)
wherein A is a carboxy group or is absent; X is a polyol, Y is —C(═O)—, —C(═S)—, or is absent; Z is O, S or NH; and R₁ is a polyether, wherein one or more hydroxy groups of the polyol are acylated with a fatty acid residue.

16. The complex of claim 15 wherein the polyol has from about 2 carbons to about 20 carbons.

17. The complex of claim 15, wherein the polyol is substituted with one or more carboxy groups.

18. The complex of claim 15, wherein the polyol is mucic acid, malic acid, citromalic acid, alkylmalic acid, hydroxy derivatives of glutaric acid, alkyl glutaric acids, tartaric acid, or citric acid.

19. The complex of claim 15, wherein the polyether is a poly(alkylene oxide) having between about 2 and about 150 repeating units.

20. The complex of claim 15, wherein the polyether is linked to the polyol through an ester, thioester, or amide linkage.

21. The complex of claim 15, wherein the polyether is a methoxy terminated poly(ethylene glycol).

22. The complex of claim 15, wherein the fatty acids comprise from about 2 to about 24 carbon atoms.

23. The complex of claim 1, wherein the AM is a compound of the following formula: 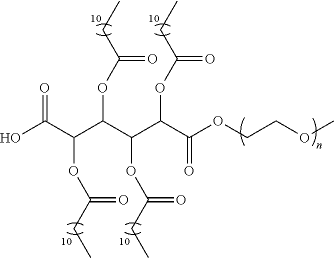
wherein n is an integer between 100 and 120.

24. A composition comprising the complex of claim 1.

25. A pharmaceutical composition comprising the complex of claim 10 and a suitable carrier.

26. A method for delivering a therapeutic agent to an animal in need of treatment with the agent, comprising administering the complex of claim 10 to the animal.

27. A method of making an amphiphilic macromolecule (AM)-lipid complex, comprising:
combining lipids and AMs in a solvent;
removing the solvent by evaporation to prepare a film;
drying the film; and
hydrated the film to produce a composition that comprises the complex.

28. The method of claim 27, further comprising filtering the composition to separate the complex.

29. A method of making an amphiphilic macromolecule (AM)-lipid complex, comprising:
adding the AM to a solution that comprises vesicles formed by the lipids so as form the AM-lipid complex.
